(19) United States
(12) Patent Application Publication
(10) Pub. No.: US 2021/0369247 A1
(43) Pub. Date: Dec. 2, 2021

(54) MEDICAL IMAGE DIAGNOSIS APPARATUS, MEDICAL IMAGE PROCESSING APPARATUS AND MEDICAL IMAGE PROCESSING METHOD

(71) Applicant: CANON MEDICAL SYSTEMS CORPORATION, Otawara-shi (JP)

(72) Inventors: Yu IGARASHI, Utsunomiya (JP); Masaki WATANABE, Utsunomiya (JP); Yasunori HONJO, Utsunomiya (JP); Tomohisa IMAMURA, Nasushiobara (JP); Tetsuya KAWAGISHI, Nasushiobara (JP)

(73) Assignee: CANON MEDICAL SYSTEMS CORPORATION, Otawara-shi (JP)

(21) Appl. No.: 17/333,410

(22) Filed: May 28, 2021

(30) Foreign Application Priority Data
May 29, 2020 (JP) 2020-094254

(51) Int. Cl. A61B 8/00; A61B 8/08

(52) U.S. Cl. CPC A61B 8/543; A61B 8/08; A61B 8/5207; A61B 8/5284; A61B 8/4245

(57) ABSTRACT

A medical image diagnosis apparatus according to an embodiment includes processing circuitry configured to acquire signals from a subject over time, calculate a first similarity representing a similarity of the signals between frames with respect to each of a plurality of frames and a plurality of positions and calculate a second similarity representing a similarity of change of the first similarity over time between the positions, and output the second similarity.

### CROSS-REFERENCE TO RELATED APPLICATIONS

This application is based upon and claims the benefit of priority from Japanese Patent Application No. 2020-094254, filed on May 29, 2020; the entire contents of which are incorporated herein by reference.

### FIELD

Embodiments described herein relate generally to a medical image diagnosis apparatus, a medical image processing apparatus and a medical image processing method.

### BACKGROUND

In a diagnosis using medical images, fluctuation evaluation is performed in some cases. For example, it is known that an angioma that is a benign tumor appears as fluctuation in a medical image. Thus, performing fluctuation evaluation on a part that is possibly a tumor makes it possible to determine whether the part is an angioma.

### BRIEF DESCRIPTION OF THE DRAWINGS

FIG. 1 is a block diagram illustrating an example of a configuration of an ultrasound diagnosis apparatus according to a first embodiment;

FIG. 2 is a diagram illustrating an example of ultrasound images according to the first embodiment;

FIG. 3 is a diagram illustrating an example of first similarities according to the first embodiment;

FIG. 4A is a diagram for explaining a process of calculating a second similarity according to the first embodiment;

FIG. 4B is a diagram for explaining the process of calculating a second similarity according to the first embodiment;

FIG. 4C is a diagram for explaining the process of calculating a second similarity according to the first embodiment;

FIG. 5A is a diagram illustrating an example of correlation curves according to the first embodiment;

FIG. 5B is a diagram illustrating an example of correlation curves according to the first embodiment;

FIG. 6 is a diagram illustrating an example of a process of calculating a second similarity according to the first embodiment;

FIG. 7 is a diagram illustrating an example a color image according to the first embodiment;

FIG. 8 is a diagram illustrating an example of a process of generating a color image according to the first embodiment;

FIG. 9 is a diagram illustrating an example of a process of generating color images according to the first embodiment;

FIG. 10 is a flowchart for explaining a sequence flow of a process performed by the ultrasound diagnosis apparatus according to the first embodiment;

FIG. 11 is a diagram illustrating an example of a process of generating a color image according to a second embodiment;

FIG. 12 is a block diagram illustrating an example of a configuration of a medical image processing system according to the second embodiment.

### DETAILED DESCRIPTION

The medical image diagnosis apparatus according to the embodiment includes processing circuitry. The processing circuitry is configured to acquire signals from a subject over time, calculate a first similarity representing a similarity of the signals between frames with respect to each of a plurality of frames and a plurality of positions and calculate a second similarity representing a similarity of change of the first similarity over time between the positions, and output the second similarity.

With reference to the accompanying drawings, embodiments of a medical image diagnosis apparatus and a medical image processing apparatus will be described in detail below.

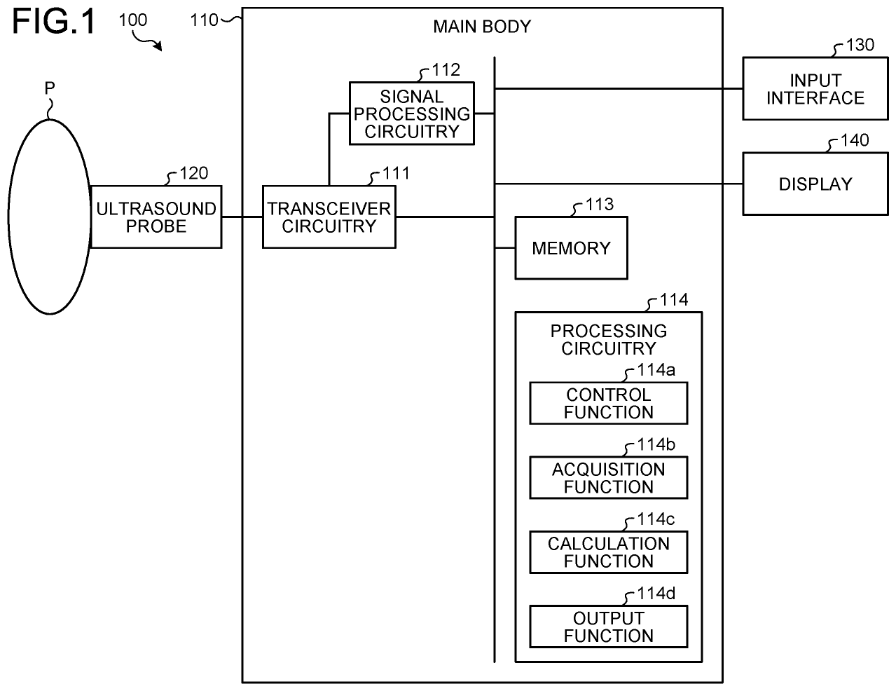

In the embodiment, an ultrasound diagnosis apparatus 100 illustrated in FIG. 1 will be described as an example of the medical image diagnosis apparatus. FIG. 1 is a block diagram illustrating an example of a configuration of the ultrasound diagnosis apparatus 100 according to the first embodiment. For example, the ultrasound diagnosis apparatus 100 includes a main body 110, an ultrasound probe 120, an input interface 130 and a display 140. The ultrasound probe 120, the input interface 130 and the display 140 are connected to the main body 110 such that they can communicate with the main body 110. A subject P is not included in the ultrasound diagnosis apparatus 100.

The ultrasound probe 120 includes a plurality of transducers (piezoelectric transducers) and the transducers generate ultrasound based on a drive signal that is supplied from transceiver circuitry 111 that the main body 110 described below includes. The transducers of the ultrasound probe 120 receives reflected waves from the subject P and converts the reflected waves into electric signals. The ultrasound probe 120 includes matching layers that are formed in the transducers and a backing member that prevents backward transmission of the ultrasound from the transducers.

When ultrasound is transmitted from the ultrasound probe 120 to the subject P, the transmitted ultrasound is reflected by a surface with discontinuity in acoustic impedance in body tissue of the subject P and is received as reflected wave signals (echo signals) by the transducers that the ultrasound probe 120 includes. The amplitude of the received reflected wave signals depends on the difference in acoustic impedance on the surface with discontinuity by which the ultrasound is reflected. Reflected wave signals in the case where ultrasound pulses are reflected by a moving blood flow or the surface of a cardiac wall undergo a frequency shift because of a Doppler effect depending on the speed components of a mobile object with respect to the direction in which ultrasound is transmitted.

The type of the ultrasound probe 120 is not particularly limited. For example, the ultrasound probe 120 may be a one-dimensional ultrasound probe in which a plurality of piezoelectric transducers are arranged in a row, a one-dimensional ultrasound probe in which a plurality of piezoelectric transducers that are arranged in a row are mechanically swung, or a two-dimensional ultrasound probe in which a plurality of piezoelectric transducers are arranged two-dimensionally.

The input interface 130 receives various input operations from a user, converts the received input operations into electric signals, and outputs the electric signals to the main body 110. For example, the input interface 130 is implemented by a mouse and a keyboard, a track ball, a switch, a button, a joystick, a touch pad whose operation screen is touched to perform an input operation, a touch screen including a display screen and a touch pad that are integrated, contactless input circuitry using an optical sensor, audio input circuitry, or the like. The input interface 130 may consists of a tablet terminal capable of wirelessly communicating with the main body 110. The input interface 130 may be circuitry that receives input operations from the user by motion capture. For example, the input interface 130 is able to receive body motions, gazes, etc., as input operations by processing signals that are acquired via a tracker and images that are obtained with respect to users. The input interface 130 is not limited to one including physical operational parts, such as a mouse and a keyboard. For example, examples of the input interface 130 include electric signal processing circuitry that receives an electric signal corresponding to an input operation from an external input device arranged independently of the main body 110 and that outputs the electric signal to the main body 110.

The display 140 displays various types of information. For example, under the control of processing circuitry 114, the display 140 displays ultrasound images that are acquired from the subject P. For example, the display 140 displays results of various types of processing performed by the processing circuitry 114. The processing performed by the processing circuitry 114 will be described below. For example, the display 140 displays a graphical user interface (GUI) for receiving various instructions and settings from the user via the input interface 130. For example, the display 140 is a liquid crystal display or a cathode ray tube (CRT) display. The display 140 may be a desktop display or may consist of a tablet terminal device capable of wirelessly communicating with the main body 110.

FIG. 1 illustrates the ultrasound diagnosis apparatus 100 as one including the display 140. The ultrasound diagnosis apparatus 100 may include a projector instead of or in addition to the display 140. Under the control of the processing circuitry 114, the projector is able to perform projection on a screen, a wall, a floor the body surface of the subject P, or the like. For example, the projector is also able to perform projection on any flat surface, objet or space by projection mapping.

The main body 110 is an apparatus that acquires signals from the subject P via the ultrasound probe 120. The main body 110 is able to generate ultrasound images based on the signals that are acquired from the subject P. For example, the main body 110 includes the transceiver circuitry 111, signal processing circuitry 112, a memory 113, and the processing circuitry 114. The transceiver circuitry 111, the signal processing circuitry 112, the memory 113, and the processing circuitry 114 are connected such that they can communicate with one another.

The transceiver circuitry 111 includes a pulse generator, a transceiver delay unit, and a pulsar and supplies a drive signal to the ultrasound probe 120. The pulse generator repeatedly generates rate pulses for forming transmission ultrasound at a given rate frequency. The transmission delay unit focuses ultrasound that is generated by the ultrasound probe 120 into a beam and applies a delay per piezoelectric transducer necessary to determine transmission directionality to each rate pulse that is generated by the pulse generator. The pulsar applies a drive signal (drive pulse) to the ultrasound probe 120 at timing based on the rate pulse. In other words, the transmission delay unit freely adjusts the direction of transmission of ultrasound that is transmitted from the surfaces of the ultrasound transducers by changing the delay to be applied to each rate pulse.

The transceiver circuitry 111 has a function of being capable of instantaneously changing a transmission frequency, a transmission drive voltage, etc., in order to execute a given scan sequence based on an instruction from the processing circuitry 114 to be described below. Particularly, a change in the transmission drive voltage is realized by a linear-amplifier-type oscillator capable of instantaneously switching the value of the transmission drive voltage or a system that electrically switch between multiple power units.

The transceiver circuitry 111 includes a preamplifier, an A/D (Analog/Digital) converter, a reception delay unit, an adder, etc., and generates reflected-wave data by performing various types of processing on reflected-wave signals that are received by the ultrasound probe 120. The preamplifier amplifies the reflected-wave signals according to each channel. The A/D converter performs A/D conversion on the amplified reflected-wave signals. The reception delay unit applies a delay necessary to determine reception directionality. The adder generates reflected-wave data by performing an add operation on the reflected-wave signals that are processed by the reception delay unit. The add operation performed by the adder enhances reflected components from a direction corresponding to the directionality of reception of the reflected wave signals and accordingly an integrated beam of ultrasound transmission and reception is formed according to the reception directionality and the transmission directionality.

When scanning a two-dimensional region in the subject P, the transceiver circuitry 111 transmits ultrasound beams in two-dimensional directions from the ultrasound probe 120. The transceiver circuitry 111 generates two-dimensional reflected-wave data from the reflected-wave signal that is received by the ultrasound probe 120. When scanning a three-dimensional region of the subject P, the transceiver circuitry 111 transmits ultrasound beams in three-dimensional directions from the ultrasound probe 120.

The signal processing circuitry 112 generates data (B-mode data) in which a signal intensity at each sample point is expressed by brightness of luminance by performing logarithmic amplification, envelope detection, etc., on the reflected-wave data that is received from the transceiver circuitry 111. The B-mode data that is generated by the signal processing circuitry 112 is output to the processing circuitry 114.

The signal processing circuitry 112 generates data (Doppler data) obtained by extracting kinetic information based on the Doppler effect of the mobile object in each sample point in a scan region from the reflected-wave data that is received from the transceiver circuitry 111. Specifically, the signal processing circuitry 112 performs frequency analysis on speed information from the reflected-wave data, extracts a blood flow or tissue and contrast agent echo components based on the Doppler effect and generates data (Doppler data) obtained by extracting mobile object information, such as an average speed, dispersion, and power, at many points. The mobile object herein includes, for example, a blood flow, tissue of a cardiac wall, or the like, or a contrast agent. The kinetic information (blood flow information) obtained by the signal processing circuitry 112 is output to the processing circuitry 114. The Doppler data can be displayed in a color image as an image of, for example, an average speed image, a dispersion image, a power image or a combination thereof.

The memory 113 is, for example, implemented by a semiconductor memory device, such as a random access memory (RAM) or a flash memory, a hard disk or an optical disk. For example, the memory 113 stores programs by which circuitry contained in the ultrasound diagnosis apparatus 100 implement the functions. The memory 113 stores various types of data acquired by the ultrasound diagnosis apparatus 100. For example, the memory 113 stores the B-mode data and the Doppler data that are generated by the signal processing circuitry 112. For example, the memory 113 stores ultrasound images that are generated by the processing circuitry 114 based on the B-mode data and the Doppler data. The memory 113 is able to store various types of data, such as diagnosis information (for example, a patient ID and an opinion of a doctor), a diagnosis protocol, and a body mark. The memory 113 may be able to be implemented by a group of servers (clouds) that are connected to the ultrasound diagnosis apparatus 100 via the network.

The processing circuitry 114 executes a control function 114a, an acquisition function 114b, a calculation function 114c, and an output function 114d, thereby controlling overall operations of the ultrasound diagnosis apparatus 100. The acquisition function 114b is an example of an acquisition unit. The calculation function 114c is an example of a calculation unit, and the output function 114d is an example of an output unit.

For example, the processing circuitry 114 reads a program corresponding to the control function 114a from the memory 113 and executes the program, thereby controlling various functions, such as the acquisition function 114b, the calculation function 114c and the output function 114d, based on various input operations that are received from the user via the input interface 130.

For example, the processing circuitry 114 reads a program corresponding to the acquisition function 114b from the memory 113 and executes the program, thereby acquiring signals from the subject P. For example, the acquisition function 114b acquires B-mode data and Doppler data from the subject P by controlling the transceiver circuitry 111 and the signal processing circuitry 112.

The acquisition function 114b may perform a process of generating ultrasound images based on signals that are acquired from the subject P. For example, based on the B-mode data, the acquisition function 114b generates a B-mode image in which the intensity of the reflected-waves are expressed by luminance. For example, based on the Doppler data, the acquisition function 114b generates a Doppler image representing the mobile object information. Note that a Doppler image is speed image data, dispersion image data, power image data, or a combination of these sets of data.

For example, by performing scan conversion on the B-mode data and the Doppler data, the acquisition function 114b generates an ultrasound image. In other words, the acquisition function 114b generates an ultrasound image by converting a scanning line signal row of ultrasound scanning into a scan line signal row of a video format represented by TV, or the like (scan conversion). For example, the acquisition function 114b generates an ultrasound image by performing coordinate transformation according to the mode of ultrasound scanning performed by the ultrasound probe 120.

The acquisition function 114b may perform various types of image processing on ultrasound images. For example, using an ultrasound images corresponding to multiple frames, the acquisition function 114b performs image processing of regenerating a luminance average image (smoothing processing) and image processing (edge enhancement processing) using a differential filter in the image. For example, the acquisition function 114b synthesizes supplementary information (such as character information on various parameters, scale marks, and body marks) with the ultrasound image. For example, when three-dimensional image data (volume data) is generated as the ultrasound image, the acquisition function 114b generates a two-dimensional image for display by performing rendering on the volume data.

For example, the processing circuitry 114 reads a program corresponding to the calculation function 114c from the memory 113 and executes the program, thereby calculating a first similarity of the signals acquired from the subject P between frames with respect to each of multiple frames and multiple positions and furthermore calculating a second similarity representing a similarity of change of the first similarity over time between positions. For example, the processing circuitry 114 reads a program corresponding to the output function 114d from the memory 113 and executes the program, thereby outputting the second similarity that is calculated by the calculation function 114c. For example, the output function 114d controls display on the display 140 and control data transmission via the network. The process performed by the calculation function 114c and the output function 114d will be described below.

The “similarity” like the first similarity and the second similarity may be any one of an index indicating a degree of similarity and an index indicating a degree of dissimilarity. In other words, the similarity may be defined such that the higher the similarity is the more the value of the similarity increases and such that the lower the similarity is the more the value of the similarity decreases.

In the ultrasound diagnosis apparatus 100 illustrated in FIG. 1, each process function is stored in the memory 113 in a form of a computer-executable program. The transceiver circuitry 111, the signal processing circuitry 112 and the processing circuitry 114 are processors that read the programs from the memory 113 and execute the programs, thereby implementing the functions corresponding to the respective programs. In other words, the transceiver circuitry 111, the signal processing circuitry 112 and the processing circuitry 114 having read the programs have the functions corresponding to the read programs.

FIG. 1 illustrates that the control function 114a, the acquisition function 114b, the calculation function 114c, and the output function 114d are implemented in the single processing circuitry 114. Alternatively, multiple independent processors may be combined to configure the processing circuitry 114 and the respective processors may execute the programs, thereby implementing the functions. The process functions that the processing circuitry 114 includes may be implemented in a manner that the process functions are distributed into multiple processing circuits or may be integrated into a single processing circuit as appropriate.

The processing circuitry 114 may implement the functions, using a processor of an external device that is connected via the network. For example, the processing circuitry 114 reads the programs corresponding to the respective functions from the memory 113 and executes the programs and uses a group of servers (clouds) that are connected to the ultrasound diagnosis apparatus 100 via the network as computation resources, thereby implementing the respective functions illustrated in FIG. 1.

The example of the configuration of the ultrasound diagnosis apparatus 100 is described above. With such a configuration, the ultrasound diagnosis apparatus 100 increases accuracy of fluctuation evaluation because of the process performed by the processing circuitry 114.

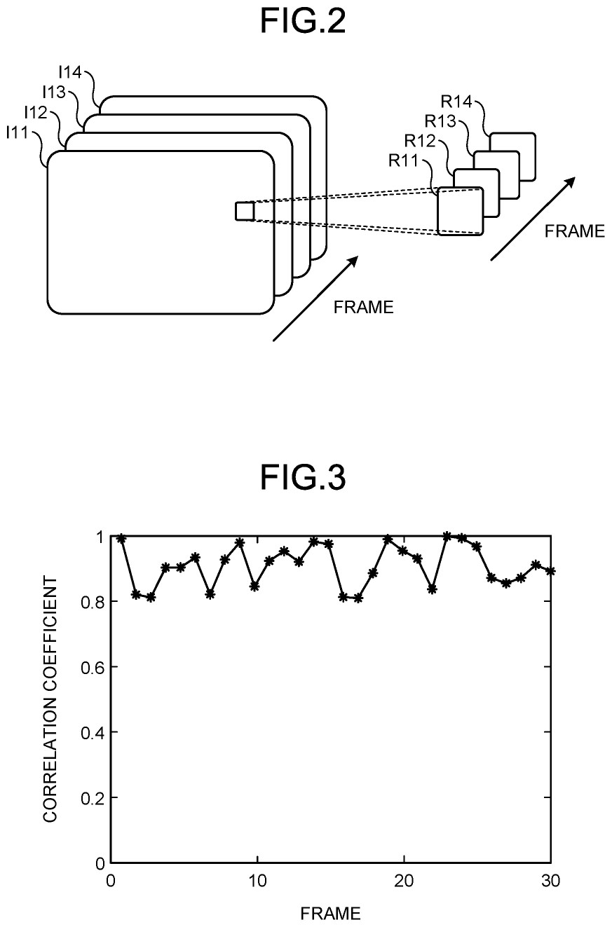

First of all, the acquisition function 114b controls the transceiver circuitry 111 and the signal processing circuitry 112, thereby acquiring signals from the subject P over time. For example, the acquisition function 114b acquires signals over time and executes an image generation process, thereby sequentially generating a B-mode image I11, a B-mode image I12, a B-mode image I13, and a B-mode image I14 that are illustrated in FIG. 2. In other words, in the case illustrated in FIG. 2, the acquisition function 114b acquires signals corresponding to multiple frames over time and generates ultrasound images of the respective frames. FIG. 2 is a diagram illustrating an example of ultrasound images according to the first embodiment.

The calculation function 114c sets an analysis region (analysis ROI). For example, the calculation function 114c receives an operation of specifying an analysis ROI from a user via the input interface 130, thereby setting an analysis ROI. For example, the output function 114d causes the display 140 to display the B-mode image I11 and the calculation function 114c receives an operation of specifying an analysis ROI from the user who have referred to the B-mode image I11. In this case, the analysis ROI that is set on the B-mode image I11 is directly applied to corresponding positions in the B-mode image I12, the B-mode image I13, and the B-mode image I14. Alternatively, the calculation function 114c may automatically set an analysis ROI based on diagnosis information, etc. Alternatively, the calculation function 114c may set the whole acquired ultrasound image for the analysis ROI.

The calculation function 114c may perform control such that the analysis ROI is changeable according to a subject to be analyzed. For example, the calculation function 114c adjusts the shape and size of the analysis ROI such that the subject to be analyzed and the area around the subject to be analyzed without local signal change are contained. In other words, the calculation function 114c adjusts the shape and size of the analysis ROI such that an area that serves as a reference of analysis is contained in addition to the subject to be analyzed.

The calculation function 114c sets a comparison region in the analysis ROI in each ultrasound image. For example, the calculation function 114c sets a kernel R11 illustrated in FIG. 2 in the B-mode image I11. The kernel R11 is a small region having a given size and a given shape and corresponds to multiple pixels in the B-mode image I11. Similarly, the calculation function 114c sets a kernel R12 in the B-mode image I12, sets a kernel R13 in the B-mode image I13, and sets a kernel R14 in the B-mode image I14. The kernel R11, the kernel R12, the kernel R13, and the kernel R14 are set in corresponding positions in the respective frames. The kernel R11, the kernel R12, the kernel R13, and the kernel R14 are examples of the comparison region.

The calculation function 114c calculates a similarity by comparing the pixels in the comparison region between frames. In other words, the calculation function 114c calculates a similarity of signals between frames. For example, the calculation function 114c calculates a correlation coefficient rxy of the image in the comparison region between adjacent frames according to Equation (1) below, where x denotes “a frame number of interest”, y denotes “the frame number of interest+1”, i denotes “an i-th pixel value”, and n denotes a total number of pixels within the comparison region.

$${r}_{x y} = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}} \qquad ( 1 )$$

For example, in the case illustrated in FIG. 2, using Equation (1) above, the calculation function 114c calculates a correlation coefficient C1 between the kernel R11 and the kernel R12, calculates a correlation coefficient C2 between the kernel R12 and the kernel R13, and calculates a correlation coefficient C3 between the kernel R13 and the kernel R14. The calculation function 114c may calculate a correlation coefficient not between adjacent frames but between frames with a given interval in between.

In other words, the calculation function 114c calculates a similarity of signals in the direction of time by comparing the pixels in the comparison region between frames. Such similarity in the time direction is also referred to as a first similarity.

The calculation function 114c may perform control such that the comparison region is changeable according to a subject to be analyzed. For example, the calculation function 114c adjusts the size of the comparison region such that the comparison region has a size equivalent to a minute change in signal. For example, the calculation function 114c adjusts the size of the comparison region to a size corresponding to contrast in density of a signal caused by fluctuation in one period. For example, the calculation function 114c adjusts the size of the comparison region to a size obtained by increasing the minute change in signal by a given magnification.

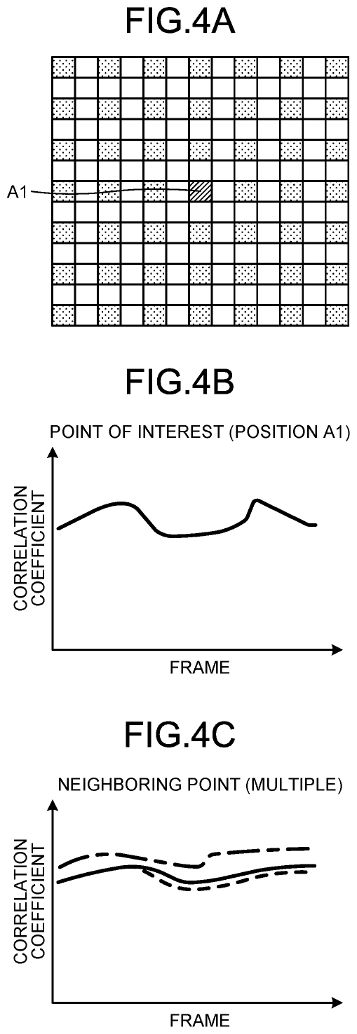

As described above, the calculation function 114c performs calculation of a first similarity on each frame. Accordingly, for example, as illustrated in FIG. 3, the first similarities can be plotted in association with frame numbers. For example, the calculation function 114c uses, as a first similarity of the frame number of the B-mode image I12, the average of the correlation coefficient C1 that is calculated between the kernel R11 and the kernel R12 and the correlation coefficient C2 that is calculated between the kernel R12 and the kernel R13. For example, the calculation function 114c uses, as a first similarity of the frame number of the B-mode image I13, the average of the correlation coefficient C2 that is calculated between the kernel R12 and the kernel R13 and the correlation coefficient C3 that is calculated between the kernel R13 and the kernel R14. FIG. 3 is a diagram illustrating an example of the first similarities according to the first embodiment.

Furthermore, shifting the comparison region in the spatial direction, the calculation function 114c repeatedly executes the process of calculating a first similarity. In other words, the calculation function 114c calculates a first similarity with respect to each position in the analysis ROI. For example, the calculation function 114c calculates a first similarity with respect to each pixel in the analysis ROI. Alternatively, the calculation function 114c may calculate a first similarity with respect to each pixel group that is a collection of multiple pixels. In other words, the calculation function 114c calculates a first similarity with respect to each frame and each position. In this case, it is possible to generate a graph like that illustrated in FIG. 3 with respect to each position in the analysis ROI.

The user is able to perform fluctuation evaluation based on the first similarities. In other words, the first similarities represent signal changes between frames and the value varies in a position where fluctuation occurs. Thus, by referring to the first similarities in a part that is possibly a tumor, the user is able to evaluate fluctuation and determine whether the part is an angioma.

During acquisition of signals corresponding to multiple frames, however, a positional shift between frames may occur due to breathing motions of the subject P or motions of the ultrasound probe 120. When such a disturbance occurs, the signal changes between frames and accordingly the value of the first similarity varies as in the case where fluctuation occurs. In other words, when fluctuation evaluation is performed based on only the first similarities, it is difficult to distinguish between fluctuation and disturbance and the change originally required to be captured is buried in some cases.

In order to deal with the positional shift between frames, motion correction between frames before calculation of first similarities is considered. For example, positional shifts each between frames can be classified into three directions that are a height direction, an orientation direction, and a depth direction and it is possible to correct positional shifts in the height direction and the orientation direction with a motion stabilizer, or the like. It is however difficult to perform motion correction on s positional shift in the depth direction. In other words, when a positional shift in the depth direction occurs, because the cross section on which signals are acquired itself changes, it is not possible to specify a corresponding position between frames. Accordingly, even when motion correction is performed as a pre-processing, at least a positional shift in the depth direction remains. The positional shift in the depth direction is also referred to as a cross-sectional shift (off-plane).

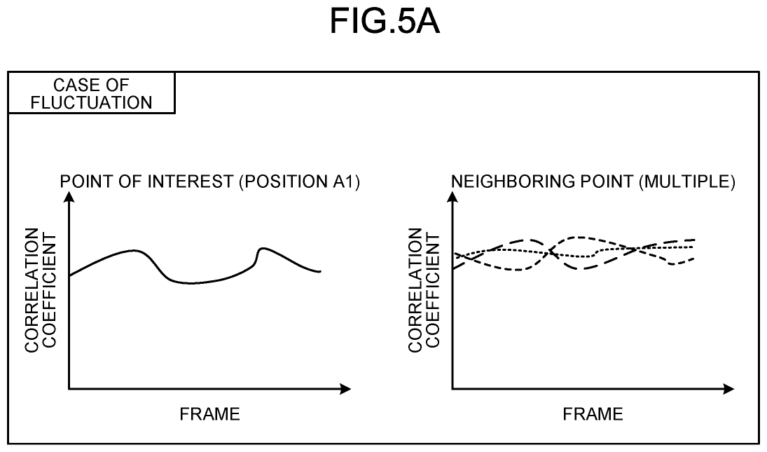

The calculation function 114c thus increases accuracy of fluctuation evaluation by further calculating a second similarity based on the first similarities. This aspect will be described below, using FIGS. 4A, 4B, and 4C. FIGS. 4A, 4B, and 4C are diagrams for explaining a process of calculating a second similarity according to the first embodiment.

For example, the calculation function 114c sets a position A1 illustrated in FIG. 4A for a point of interest and acquires changes in the first similarity over time at the point of interest. For example, the calculation function 114c plots the first similarities that are calculated with respect to the point of interest in association with the frame numbers as in the case illustrated in FIG. 3. Furthermore, by approximating the plots by a curve, thereby generating a curve illustrated in FIG. 4B. In other words, the curve illustrated in FIG. 4B represents the changes in the first similarity at the point of interest in the direction of frames. The curve representing the changes in the first similarity in the frame direction is also referred to as a correlation curve. The point of interest is also referred to as a first position.

Furthermore, the calculation function 114c generates a correlation curve with respect to each neighboring point contained in a given area neighboring to the point of interest. For example, the calculation function 114c previously sets a square area of “7 pixels×7 pixels” for the given area. In this case, as illustrated in FIG. 4A, 48 pixels neighboring to the point of interest are defined as the neighboring points. As illustrated in FIG. 4C, the calculation function 114c generates a correlation curve with respect to each of the neighboring points. The neighboring point is also referred to as a second position.

The calculation function 114c may perform control such that the given area is changeable according to the subject to be analyzed. In other words, the calculation function 114c may perform control such that the area of neighboring points is changeable according to the subject to be analyzed. It is described that there are multiple neighboring points in FIGS. 4A to 4C. Alternatively, only one neighboring point may be set.

The calculation function 114c calculates a similarity between the correlation curve that is generated with respect to the point of interest and the correlation curves that are generated with respect to the neighboring points. For example, the calculation function 114c calculates a correlation coefficient between the correlation curve of each of the multiple neighboring points and the correlation curve of the point of interest and calculates the average of the correlation coefficients.

In other words, the calculation function 114c compares the correlation curve of the point of interest with the correlation curves of different positions, thereby calculating a similarity of change of the first similarity over time in the spatial direction. The similarity in the spatial direction is also referred to as a second similarity below.

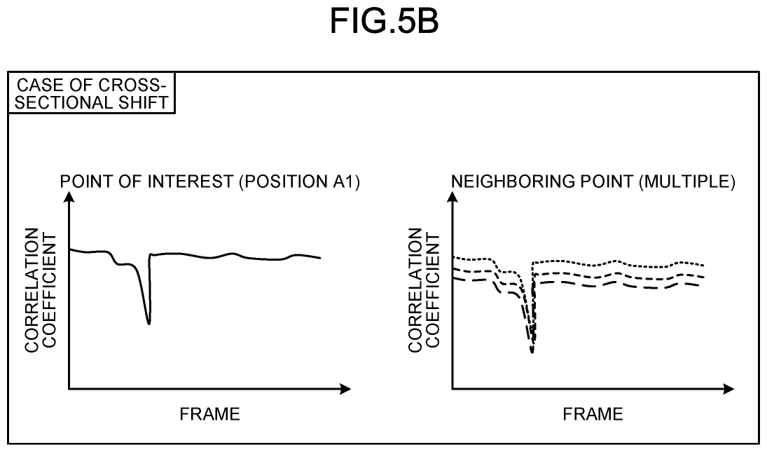

In the case where fluctuation occurs in the point of interest, when the correlation curves of the point of interest and the neighboring points are compared mutually, as illustrated in FIG. 5A, the positions and heights of peaks are irregular. In other words, in the case where there is a specific signal change locally compared to the surroundings in a tumor like an angioma, because the first similarity differs depending on the position when the correlation curves of the point of interest and the neighboring points are compared to each other, the correlation curve viewed in the time direction itself differs too depending on the site of analysis. Accordingly, when fluctuation occurs, the correlation curve of the point of interest tends not to be similar to the correlation curves of the neighboring points. Note that FIG. 5A is a diagram illustrating an example of the correlation curves according to the first embodiment.

On the other hand, when a disturbance occurs, as illustrated in FIG. 5B, variation in the frame direction occurs similarly in the correlation curves of the point of interest and the neighboring points. In other words, positional shifts occur at the same timing in any position and thus both the correlation curves of the point of interest and the neighboring points uniformly change at the same timing. That is, when a disturbance occurs, the correlation curve of the point of interest tends to be similar to the correlation curves of the neighboring points. Thus, by calculating the similarity of the correlation curves as the second similarity, it is possible to distinguish disturbance and fluctuation from each other. FIG. 5B is a diagram illustrating an example of correlation curves according to the first embodiment.

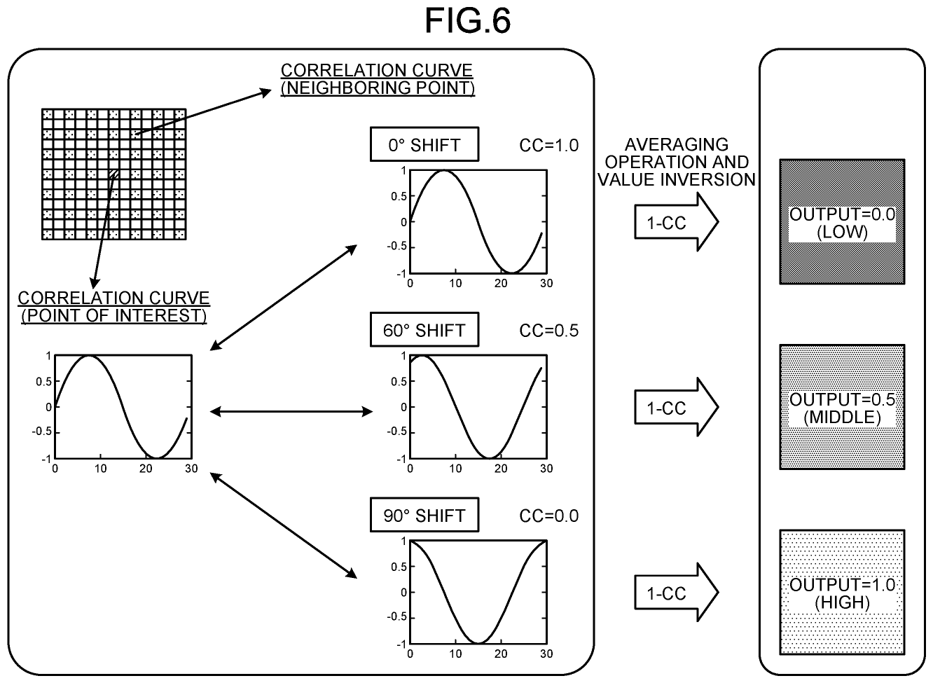

Using FIG. 6, a process of calculating a second similarity will be described in detail. FIG. 6 is a diagram illustrating an example of the process of calculating a second similarity according to the first embodiment. In FIG. 6, for the purpose of illustration, the correlation curved are represented as sine curves.

For example, when the peak position of the correlation curve of a neighboring point coincides with that of the correlation curve of the point of interest and “0° shift” applies, the calculation function 114c calculates “correlation coefficient CC=1.0”. When “60° shift” applies, the calculation function 114c calculates “correlation coefficient CC=0.5”. When “90° shift” applies, the calculation function 114c calculates “correlation coefficient CC=0.0”. The calculation function 114c calculates correlation coefficients CC with respect to the respective neighboring points and performs an averaging operation on the correlation coefficients CC.

The calculation function 114c may perform an operation of inverting a value. In other words, the case where the correlation coefficient CC is large like the case where “0° shift” applies is considered as the case where a change occurs in each position at the same timing because of a disturbance. On the other hand, the case where the correlation coefficient CC is small like the case where “60° shift” applies or “90° shift” applies is considered as the case where a local change occurs due to fluctuation. On fluctuation evaluation, because instinctive easier understanding is enabled when the value increases when the characteristics of fluctuation appears, the calculation function 114c may invert the values of the correlation coefficients CC. For example, the calculation function 114c calculates a value obtained by subtracting the value of a correlation coefficient CC from 1 as a second similarity.

As described above, the calculation function 114c calculates a second similarity by calculating correlation coefficients CC between the point of interest and the neighboring points and performing an averaging operation and a value inverting operation. For example, the calculation function 114c is able to calculate a second similarity by a “1-mean (CCi)” expression where “i” is the number of neighboring points. The calculation function 114c calculates a second similarity with respect to each position in the analysis ROI by repeating the process of calculating a second similarity while moving the position of interest in the analysis ROI.

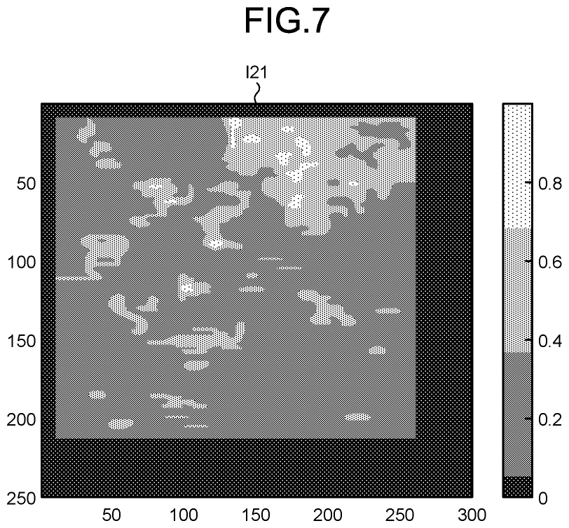

The output function 114d outputs the second similarities that are calculated by the calculation function 114c. For example, the output function 114d generates an image representing a distribution of the second similarities and outputs the image. For example, the output function 114d generates a color image illustrated in FIG. 7 by assigning colors corresponding to the magnitudes of the second similarities to the respective pixels (positions). The color herein may be any one of hue, brightness, chroma or a combination thereof. The color image is also referred to as a parametric image. The output function 114d causes the display 140 to display the generated color image. FIG. 7 is a diagram illustrating an example of the color image according to the first embodiment.

The color image may be displayed as a still image or may be displayed as a moving image. To make a still-image display, the calculation function 114c calculates second similarities on at least one frame and the output function 114d generates at least one color image and causes the display 140 to display the generated color image. To make a moving-image display, the calculation function 114c calculates second similarities on multiple frames and the output function 114d generates color images of the respective frames and causes the display 140 to display the generated color images sequentially.

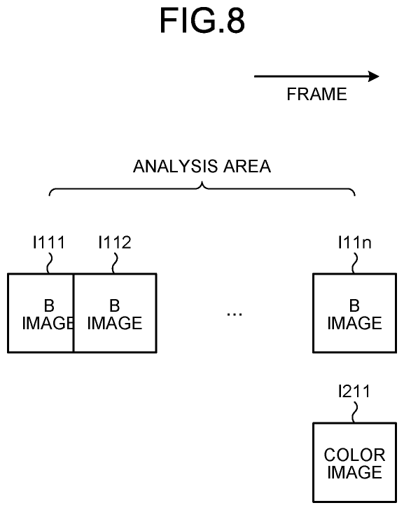

The case where a still image display is made will be described using FIG. 8. FIG. 8 is a diagram illustrating an example of a process of generating a color image according to the first embodiment. For example, as illustrated in FIG. 8, the calculation function 114c performs calculation of a first similarity on each position in an analysis ROI with respect to each of B-mode images I111 to I11n. In other words, the given number “n” presented in FIG. 8 represents an analysis area in the frame direction. The calculation function 114c generates a correlation curve representing a change in the first similarity in the frame direction with respect to each position in the analysis ROI. The calculation function 114c then calculates a second similarity representing a similarity of correlation curves between positions with respect to each position in the analysis ROI.

The output function 114d assigns colors corresponding to the magnitudes of the second similarities to the respective pixels, thereby generating a color image I211. The color image I211 illustrated in FIG. 8 is a color image whose corresponding analysis area is n frames from the B-mode image I111 to the B-mode image I11n. The output function 114d causes the display 140 to display the color image I211 as a still image.

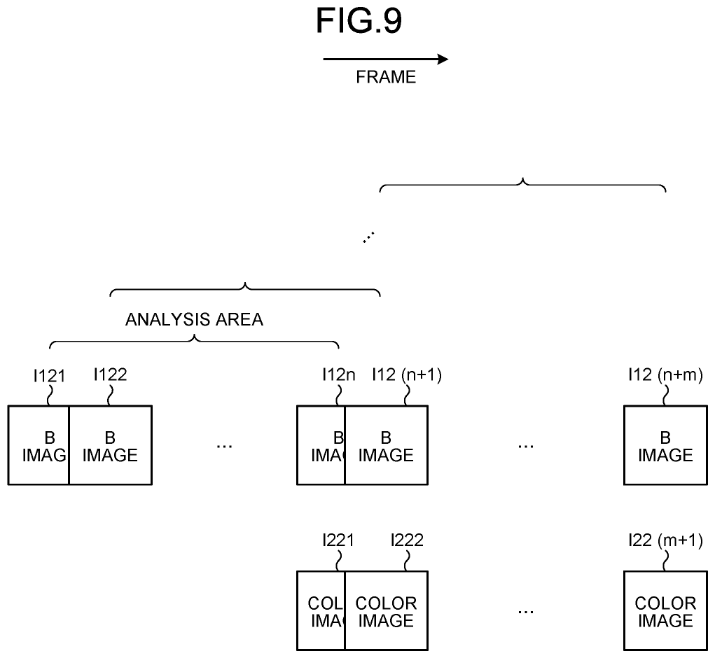

The case where a moving image display is made will be described using FIG. 9. FIG. 9 is a diagram illustrating an example of a process of generating color images according to the first embodiment. For example, as illustrated in FIG. 9, the calculation function 114c performs calculation of a first similarity on each position in an analysis ROI with respect to each of B-mode images I121 to I12n. The calculation function 114c generates a correlation curve representing a change in the first similarity in the frame direction with respect to each position in the analysis ROI. The calculation function 114c then calculates a second similarity representing a similarity of correlation curves between positions with respect to each position in the analysis ROI. The output function 114d assigns colors corresponding to the magnitudes of the second similarities to the respective pixels, thereby generating a color image I221. The color image I221 is a color image whose corresponding analysis area is n frames from the B-mode image I121 to the B-mode image I12n.

When new signals are acquired, the calculation function 114c calculates first similarities with respect to a new frame and further calculates second similarities based on the first similarities that are calculated with respect to a given number of frames from the new frame. For example, when signals are acquired newly and a B-mode image I12(n+1) is generated, the calculation function 114c performs calculation of a first similarity on each position in the analysis ROI with respect to the B-mode image I12(n+1). Based on the first similarities that are calculated with respect to n frames from the B-mode image I122 to the B-mode image I12(n+1), the calculation function 114c generates correlation curves representing a change in the first similarity in the frame direction with respect to the respective positions in the analysis ROI. The calculation function 114c calculates a second similarity representing a similarity of correlation curves between positions with respect to each position in the analysis ROI. The output function 114d assigns colors corresponding to the magnitudes of the second similarities to the respective pixels, thereby generating a color image I222. The color image I222 is a color image whose corresponding analysis area is n frames from the B-mode image I122 to the B-mode image I12(n+1).

Similarly, the calculation function 114c and the output function 114d are able to generate a color image and display the color image every time signals are acquired newly. For example, the calculation function 114c and the output function 114d are able to generate color images in real time in parallel with signal acquisition from the subject P and cause the display 140 to make a video image display.

Control may be performed such that the given number “n” presented in FIGS. 8 and 9 is changeable according to the subject to be analyzed. In other words, the calculation function 114c may perform control such that the analysis area in the frame direction is changeable according to the subject to be analyzed. For example, when the subject to be analyzed is a part that periodically moves because of heart beats, breathing, or the like, the calculation function 114c adjusts the given number “n” such that motions of the subject to be analyzed of at least one period is contained. For example, the calculation function 114c previously analyzes an analysis time required for a difference appearing in an analysis result between the subject to be analyzed and the surrounding area and adjusts the given number “n” according to the required analysis time.

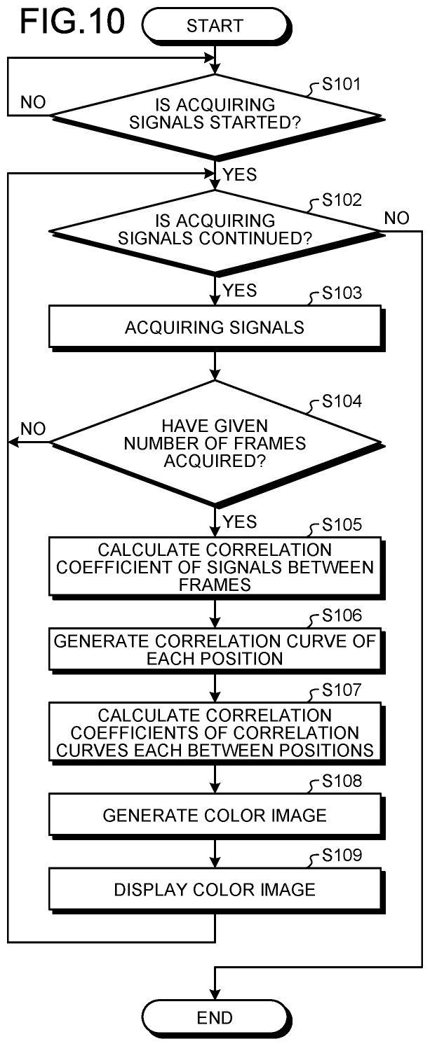

An example of procedural steps taken by the ultrasound diagnosis apparatus 100 will be described using FIG. 10. FIG. 10 is a flowchart for explaining a sequence flow of a process performed by the ultrasound diagnosis apparatus 100 according to the first embodiment. Steps S101, S102 and S103 correspond to the acquisition function 114b. Steps S104, S105, S106 and S107 correspond to the calculation function 114c. Steps S108 and S109 correspond to the output function 114d. FIG. 10 exemplifies and illustrates the case where, as in the case illustrated in FIG. 9, generation and display of color images are performed in parallel with signal acquisition from the subject.

First of all, the processing circuitry 114 determines whether to start acquiring signals from the subject P (step S101) and, when acquiring signals is not started, enters a stand-by state (NO at step S101). On the other hand, when acquiring signals is started, the processing circuitry 114 determines whether to continue acquiring signals (step S102) and, when acquiring signals is continued, controls the transceiver circuitry 111 and the signal processing circuitry 112 and acquires signals from the subject P (step S103).

The processing circuitry 114 then determines whether a given number of frames have been acquired (step S104). For example, in the cases illustrated in FIGS. 8 and 9, the given number n of frames are the analysis area and it is not possible to generate a color image until signals of n frames are acquired. Thus, when the given number of frames have not been acquired (NO at step S104), the processing circuitry 114 moves to step S102 again and continue acquiring signals.

On the other hand, when the given number of frames have been acquired (YES at step S104), the processing circuitry 114 calculates a correlation coefficient of signals between frames with respect to each position in an analysis ROI (step S105). In other words, the processing circuitry 114 calculate first similarities. The processing circuitry 114 generates correlation curves with respect to the respective positions in the analysis ROI (step S106) and calculates correlation coefficients of the correlation curves each between positions (step S107). In other words, the processing circuitry 114 calculates a second similarity.

The processing circuitry 114 generates a color image by assigning colors corresponding to the magnitudes of second similarities to the respective pixels (step S108) and causes the display 140 to display the generated color image (step S109). After step S109, the processing circuitry 114 moves to step S102 again and determines whether to continue acquiring signals. When acquiring signals is continued, the processing circuitry 114 executes the process from step S103 to step S109 again. In other words, while continuing acquiring signals, the processing circuitry 114 updates the color image based on newly acquired signals and causes the display 140 to make a moving image display. On the other hand, when acquiring signals is not continued (NO at step S102), the processing circuitry 114 ends the process.

As described above, according to the first embodiment, the acquisition function 114b acquires signals from the subject P over time. The calculation function 114c calculates first similarities each representing a similarity of signals between frames and calculates second similarities each representing a similarity of change of the first similarity over time between positions. The output function 114d outputs the second similarities. Accordingly, the ultrasound diagnosis apparatus 100 according to the first embodiment is able to increase accuracy of fluctuation evaluation.

In other words, the ultrasound diagnosis apparatus 100 outputs numeric values representing fluctuation, thereby enabling quantitative fluctuation evaluation. Furthermore, there is the case where, when fluctuation evaluation is performed based on similarities in signals between frames, a change to be originally required to be captured is buried due to the effect of disturbance, such as a cross-sectional shift. The ultrasound diagnosis apparatus 100 calculates first similarities each representing a similarity of signals between frames and calculates second similarities each representing a similarity of change of the first similarity over time between positions. This enables the ultrasound diagnosis apparatus 100 to distinguish fluctuation from disturbance and increase accuracy of fluctuation evaluation.

The first embodiment has been described and various different modes may be carried out in addition to the above-described embodiment.

For example, the first embodiment presents, as for the case where color images are displayed as a moving image fr, the example illustrated in FIG. 9. In other words, FIG. 9 illustrates the case where a color image is generated and displayed every time a new frame is acquired. Embodiments are however not limited to this.

For example, the calculation function 114c and the output function 114d may generate and display color images every time a given number of frames are acquired.

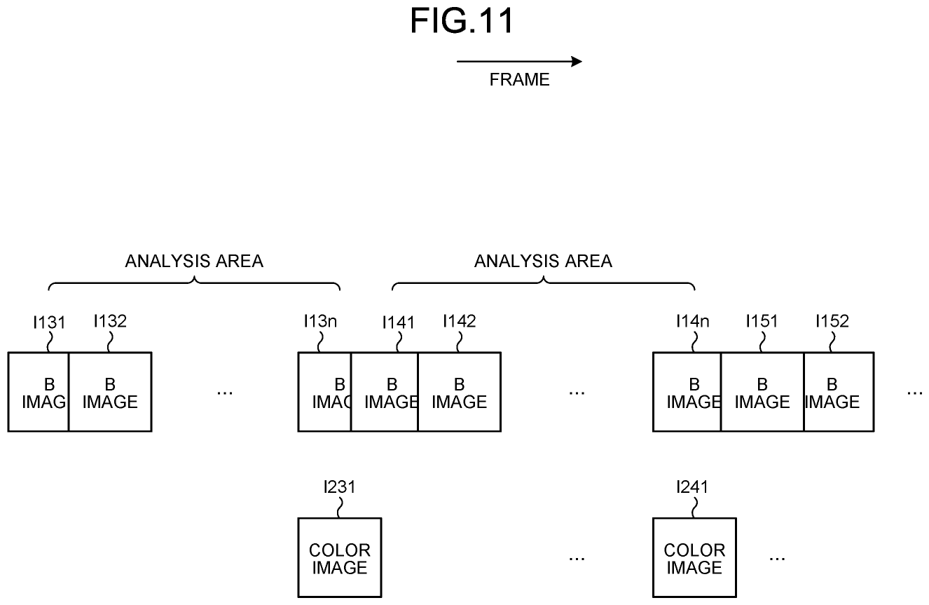

Specifically, as illustrated in FIG. 11, the calculation function 114c performs calculation of a first similarity on each position in an analysis ROI with respect to each of B-mode images I131 to I13n. The calculation function 114c generates a correlation curve representing a change in the first similarity in the frame direction with respect to each position in the analysis ROI. The calculation function 114c then calculates a second similarity representing a similarity in correlation curves between positions with respect to each position in the analysis ROI. The output function 114d assigns colors corresponding to the magnitudes of the second similarities to the respective pixels, thereby generating a color image I231. The color image I231 is a color image whose corresponding analysis area is n frames from the B-mode image I131 to the B-mode image I13n. The output function 114d causes the display 140 to display the color image I231. FIG. 11 is a diagram illustrating an example of a process of generating a color image according to a second embodiment.

Similarly, the calculation function 114c performs calculation of a first similarity on each position in an analysis ROI with respect to each of B-mode images I141 to I14n. The calculation function 114c generates a correlation curve representing change of the first similarity in the frame direction with respect to each position in the analysis ROI. The calculation function 114c then calculates a second similarity representing a similarity of correlation curves between positions with respect to each position in the analysis ROI. The output function 114d assigns colors corresponding to the magnitudes of the second similarities to the respective pixels, thereby generating a color image I241. The color image I241 is a color image whose corresponding analysis area is n frames from the B-mode image I141 to the B-mode image I14n. The output function 114d causes the display 140 to display the color image I241 instead of the color image I231.

In the case illustrated in FIG. 11, compared to the case illustrated in FIG. 9, the frequency of generation of a color image lowers and accordingly the frame rate lowers as a moving image. Note that, in the case illustrated in FIG. 11, compared to the case illustrated in FIG. 9, frequency of calculation of a second similarity lowers and this enables reduction in load of calculation. The output function 114d may switch the display mode in FIG. 9 or the display mode in FIG. 11 according to an instruction from the user.

In the above-described embodiment, the case where color image display is performed in parallel with signal acquisition is described. In other words, in the above-described embodiment, the process in real time is described; however, embodiments are not limited to this.

For example, the acquisition function 114b acquires signals from the subject P over time, generates ultrasound images corresponding to multiple frames, and saves the ultrasound images in the memory 113, an external image storage device, or the like. The calculation function 114c then reads the saved ultrasound images, for example, in response to a request from the user and calculates first similarities and second similarities. The output function 114d then generates a color image representing the distribution of the second similarities and causes the display 140 to display the color image.

Alternatively, the output function 114d generates a color image representing distribution of second similarities and saves the color image in the memory 113, the external image storage device, or the like. The output function 114d then reads the saved color image, for example, in response to a request from the user and causes the display 140 to display the color image.

In the above-described embodiment, the case where the output function 114d causes the display 140 to display the generated color image; however, embodiments are not limited to this. For example, the output function 114d may transmit the generated color image to an external device. In this case, the user is able to refer to the color image, for example, on a display that the external device includes.

In the above-described embodiment, the case where the color image representing the distribution of the second similarities is generated and output is described as an example of the process of outputting second similarities; however, embodiments are not limited to this. For example, the output function 114d may output the calculated second similarities in a graph, a table or a text. For example, the output function 114d may generate a graph in which sets of coordinates of the positions in the analysis ROI are associated with the second similarities and cause the display 140 to display the graph.

In the above-described embodiment, the correlation coefficients according to Equation (1) are described as the first similarities; however, embodiments are not limited to this. For example, the calculation function 114c may calculate a SAD (Sum of Absolute Difference) or a SSD (Sum of Squared Difference) as the first similarity.

In another example of the process of calculating a first similarity, the calculation function 114c first of all performs a subtraction operation between images at a pre-set frame interval on B-mode images corresponding to multiple frames and generates differential images corresponding to multiple frames. In the B-mode images, background components are sometimes mixed in addition to fluctuation components originating from an angioma. The background components include, for example, fluctuation originating from various factors, such as fluctuation originating from liver tissue, fluctuation resulting from manipulation by the user, fluctuation resulting from the apparatus performance, and fluctuation of speckles. The calculation function 114c is able to remove the background components by performing the subtraction operation between the images.

The calculation function 114c takes an absolute value of the pixel value of each pixel with respect to each of the differential images. In other words, the differential values of the respective pixels contained in the differential image contain negative values and the calculation function 114c converts the negative values into positive values. The calculation function 114c then calculates an integration value obtained by integrating the absolute value of each pixel and absolute values of neighboring pixels. For example, using a kernel (small area), the calculation function 114c integrates the absolute value of each pixel and the absolute values of the surrounding pixels. The calculation function 114c calculates the average of the pixel value of each pixel and the pixel values of the surrounding pixels with respect to each of the B-mode images. For example, using kernels, the calculation function 114c calculates an average of a pixel value of each pixel and pixel values of neighboring pixels.

The calculation function 114c calculates a quotient obtained by dividing the integral by the average. The calculation function 114c is able to calculate a quotient with respect to each position in the analysis ROI in each frame. The calculation function 114c then integrates the quotients of the each position in the frame direction, thereby calculating an index value. For example, when the quotients corresponding to N frames are integrated, the calculation function 114c is able to calculate an index value based on the signals corresponding to N frames in the past in each frame and each position. The greater the signal resulting from fluctuation varies between frames, the higher the index value is. In other words, the index values are an example of the first similarities each representing a similarity of signals between frames.

In the above-described embodiment, the case where a similarity to a correlation curve in a different position is calculated as a second similarity is described; however, embodiments are not limited thereto. For example, the calculation function 114c may generate a scatter plot or a line graph obtained by plotting first similarities in association with frame numbers with respect to each position in the analysis ROI and calculate a similarity to a scatter plot or a line graph of a different point may be calculated as the second similarity. For example, the calculation function 114c may calculate a statistical value representing change of the first similarity over time with respect to each position in the analysis ROI and calculate a similarity to a statistical value of a different position may be calculated as the second similarity.

The acquisition function 114b, the calculation function 114c and the output function 114d are able to further perform various types of processing that are not illustrated in the above-described embodiment. For example, the calculation function 114c may perform various types of image processing on B-mode images prior to calculation of first similarities. For example, the calculation function 114c applies a low-pass filter in the frame direction, a medial filter in the spatial direction, etc., to B-mode images corresponding to multiple frames. Thus, the calculation function 114c is able to reduce various types of noise, such as spike noise and speckle noise, and accurately calculate first similarities and second similarities based on the first similarities.

In the above-described embodiment, the case where fluctuation of an angioma is evaluated is described; however, embodiments are not limited to this. In other words, the evaluation is applicable not only to angiomas but also to change in tissue presenting fluctuation.

In the above-described embodiment, it is described that B-mode images are generated and first similarities and second similarities are calculated based on B-mode images; however, embodiments are not limited to this. For example, the calculation function 114c may be able to calculate first similarities and second similarities based on other ultrasound images, such as color Doppler images and elastography, shear wave elastography (SWE) or attenuation imaging images.

In the above-described embodiment, it is described that first similarities and second similarities are calculated based on ultrasound images; however, embodiments are not limited to this. For example, the calculation function 114c may calculate first similarities and second similarities based on B-mode data. In other words, the calculation function 114c is able to perform a process of calculating first similarities and second similarities based on images or based on data before the image generation process.

In the above-described embodiment, the process on signals that are acquired by the ultrasound diagnosis apparatus 100 is described; however, embodiments are not limited to this. For example, the process is similarly applicable to signals that are acquired by a medical image diagnosis apparatus of another type, such as a photo-ultrasound diagnosis apparatus (photo acoustic imaging apparatus), an X-ray diagnosis apparatus, an X-ray CT (Computed Tomography) apparatus, an MRI (Magnetic Resonance Imaging) apparatus, a SPECT (Single Photon Emission Computed Tomography) apparatus, or a PET (Positron Emission computed Tomography) apparatus.

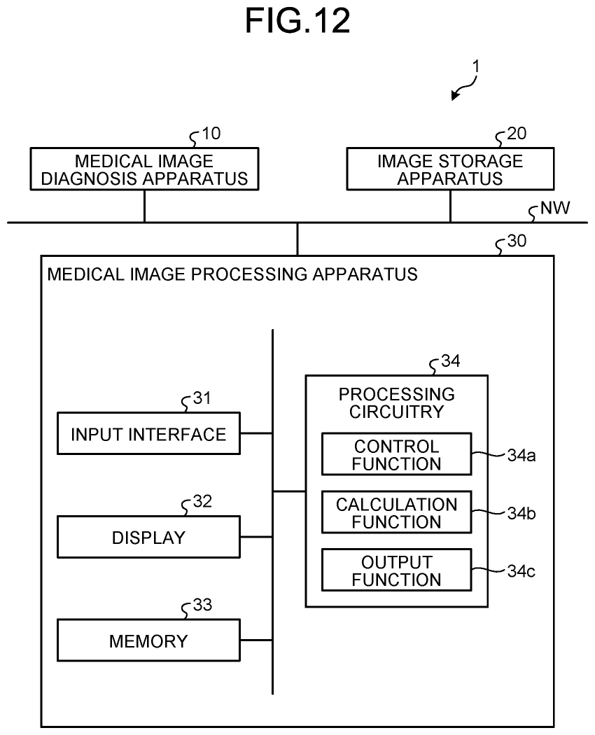

In the above-described embodiment, it is described that the processing circuitry 114 that the ultrasound diagnosis apparatus 100 includes executes the calculation function 114c and the output function 114d. In other words, in the above-described embodiment, it is described that the medical image diagnosis apparatus that executes acquisition of signals from the subject P executes the calculation function 114c and the output function 114d; however, embodiments are not limited to this. A apparatus different from the medical image diagnosis apparatus may execute a function corresponding to the calculation function 114c and the output function 114d. This aspect will be described below using FIG. 12. FIG. 12 is a block diagram illustrating an example of a configuration of a medical image processing system 1 according to the second embodiment.

The medical image processing system 1 illustrated in FIG. 12 includes a medical image diagnosis apparatus 10, an image storage apparatus 20 and a medical image processing apparatus 30. For example, the medical image diagnosis apparatus 10, the image storage apparatus 20 and the medical image processing apparatus 30 are connected with one another via a network NW. The medical image diagnosis apparatus 10, the image storage apparatus 20 and the medical image processing apparatus 30 may be set in any sites as long as they are connectable with one another via the network NW. For example, the medical image diagnosis apparatus 10, the image storage apparatus 20 and the medical image processing apparatus 30 may be set in different facilities. In other words, the network NW may consist of a closed local area network in a facility or may be a network via the Internet.

The medical image diagnosis apparatus 10 is an apparatus that executes signal acquisition from the subject P. For example, the medical image diagnosis apparatus 10 is the ultrasound diagnosis apparatus 100 in FIG. 1. Alternatively, the medical image diagnosis apparatus 10 may be a photo-ultrasound diagnosis apparatus, an X-ray diagnosis apparatus, an X-ray CT apparatus, an MRI apparatus, a SPECT apparatus, or a PET apparatus.

The medical image diagnosis apparatus 10 may transmit signals that are acquired from the subject P to the image storage apparatus 20 or the medical image processing apparatus 30. The medical image diagnosis apparatus 10 may generate and transmit an image or may transmit data before the image generation process. For example, the medical image diagnosis apparatus 10 may transmit B-mode images or B-mode data.

The image storage apparatus 20 stores various types of data acquired by the medical image diagnosis apparatus 10. The image storage apparatus 20 may store images, such as B-mode images, or store data before the image generation process, such as B-mode data. For example, the image storage apparatus 20 is a server of a PACS (Picture Archiving and Communication System).

The medical image processing apparatus 30 is an apparatus that executes functions corresponding to the calculation function 114c and the output function 114d. For example, as illustrated in FIG. 12, the medical image processing apparatus 30 includes an input interface 31, a display 32, a memory 33 and processing circuitry 34. The input interface 31, the display 32 and the memory 33 can be configured similarly to the input interface 130, the display 140, and the memory 113 in FIG. 1.

By executing a control function 34a, a calculation function 34b, and an output function 34c, the processing circuitry 34 controls entire operations of the medical image processing apparatus 30. The calculation function 34b is an example of the calculation unit. The output function 34c is an example of the output unit.

For example, the processing circuitry 34 reads a program corresponding to the control function 34a from the memory 33 and executes the program, thereby controlling various functions including the calculation function 34b and the output function 34c based on various input operations that are received from a user via the input interface 31.

For example, the processing circuitry 34 reads a program corresponding to the calculation function 34b from the memory 33 and executes the program, thereby executing the same function as the calculation function 114c in FIG. 1. Specifically, the calculation function 34b first of all acquires signals that are acquired from the subject P over time. For example, the calculation function 34b acquires, via the network NW, the signals that are acquired by the medical image diagnosis apparatus 10 and stored in the image storage apparatus 20. Alternatively, the calculation function 34b may acquire signals directly from the medical image diagnosis apparatus 10 not via the image storage apparatus 20. The calculation function 34b calculates a first similarity of the signals that are acquired from the subject P over time between frames and calculates a second similarity representing a similarity of change of the first similarity over time between positions.

For example, the processing circuitry 34 reads a program corresponding to the output function 34c from the memory 33 and executes the program, thereby executing the same function as the output function 114d in FIG. 1. In other words, the output function 34c outputs the second similarities that are calculated by the calculation function 34b. For example, the output function 34c generates an image representing a distribution of the second similarities and causes the display 32 to display the image. In another example, the output function 34c generates an image representing a distribution of the second similarities and transmits the image to an external device.

In the medical image processing apparatus 30 illustrated in FIG. 1, each process function is stored in a form of a computer-executable program in the memory 33. The processing circuitry 34 is a processor that reads the programs from the memory 33 and executes the programs, thereby implementing the functions corresponding to the respective programs. In other words, the processing circuitry 34 having read the programs has the functions corresponding to the read programs.

FIG. 12 illustrates that the single processing circuitry 34 implements the control function 34a, the calculation function 34b, and the output function 34c. Alternatively, the processing circuitry 34 may consist of a combination of multiple independent processors and the processors may execute the programs, respectively, thereby implementing the functions. The processing functions that the processing circuitry 34 includes may be distributed to multiple processing circuits or integrated into a single processing circuit as appropriate.

The processing circuitry 34 may implement the functions, using a processor of an external device that is connected via the network NW. For example, the processing circuitry 34 reads the programs corresponding to the respective functions from the memory 33 and executes the programs and uses a group of servers (clouds) that are connected to the medical image processing apparatus 30 via the network NW as calculation resources, thereby implementing each of the functions illustrated in FIG. 12.

The word “processor” used in the descriptions given above refers to, for example, a circuit, such as a CPU (Central Processing Unit), a GPU (Graphics Processing Unit), an ASIC (Application Specific Integrated Circuit), or a programmable logic device (for example, a SPLD (Simple Programmable Logic Device), a CPLD (Complex Programmable Logic Device) or a FPGA (Field Programmable Gate Array)). When the processor is, for example, a CPU, the processor reads programs that are saved in a memory and executes the programs, thereby implementing the functions. On the other hand, when the processor is, for example, an ASIC, instead of saving the programs in the memory, the functions are directly incorporated as a logic circuit in the circuit of the processor. Each processor of the embodiment is not limited to the case where each processor is configured as a single circuit. Multiple independent circuits may be combined to configure a single processor and the functions may be implemented. Furthermore, the components in each drawing may be integrated into one processor and functions thereof may be implemented.

FIG. 1 illustrates that the single memory 113 stores the programs corresponding to the respective process functions of the processing circuitry 114. FIG. 12 illustrates that the single memory 33 stores the programs corresponding to the respective process functions of the processing circuitry 34; however, embodiments are not limited to this. For example, the multiple memories 113 may be arranged in a distributed manner and the processing circuitry 114 may be configured to read a corresponding program from the individual memory 113. Similarly, the multiple memories 33 may be arranged in a distributed manner and the processing circuitry 34 may be configured to read a corresponding program from the individual memory 33. Instead of saving the programs in a memory, the programs may be directly incorporated in circuitry of a processor. In this case, the processor reads the programs that are incorporated in the circuitry and executes the programs, thereby implementing the functions.

Each of the components of each apparatus according to the above-described embodiments is a functional idea and thus need not necessarily be configured physically as unillustrated in the drawings. In other words, specific modes of distribution and integration of the apparatus are not limited to those illustrated in the drawings, and all or part of the apparatus may be configured in a distributed or integrated manner functionally or physically in any unit according to various types of load and the situation in which the apparatus are used. Furthermore, all or any of the process functions implemented by the respective apparatus may be implemented by a CPU and a program that is analyzed and executed by the CPU or may be implemented as hardware using a wired logic.

The medical image processing method described in the above-described embodiments can be implemented by executing a medical image processing program that is prepared in advance with a computer, such as a personal computer or a work station. The medical image processing program can be distributed via a network, such as the Internet. The medical image processing program may be recorded in a computer-readable and non-transient recording medium, such as a hard disk, a flexible disk (FD), a CD-ROM, a MO or a DVD, and may be read from the recording medium by the computer and thus executed.

According to at least one of the embodiments described above, it is possible to increase accuracy of fluctuation evaluation.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnosis apparatus comprising processing circuitry configured to
acquire signals from a subject over time,
calculate a first similarity representing a similarity of the signals between frames with respect to each of a plurality of frames and a plurality of positions and calculate a second similarity representing a similarity of change of the first similarity over time between the positions, and
output the second similarity.

2. The medical image diagnosis apparatus according to claim 1, wherein the processing circuitry is configured to generate a correlation curve representing a change of the first similarity in a frame direction as the change of the first similarity over time and calculates a similarity to the correlation curve of a different position as the second similarity.

3. The medical image diagnosis apparatus according to claim 1, wherein the processing circuitry is configured to calculate, as the second similarity, a similarity between the change of the first similarity over time that is calculated with respect to a first position and the change of the first similarity over time that is calculated with respect to a second position that is contained in a given area neighboring to the first position.

4. The medical image diagnosis apparatus according to claim 3, wherein the processing circuitry is configured to perform control such that the given area is changeable according to a subject to be analyzed.

5. The medical image diagnosis apparatus according to claim 1, wherein the processing circuitry is configured to set an analysis region on which the first similarity is calculated and calculate the first similarity with respect to a position that is contained in the analysis region.

6. The medical image diagnosis apparatus according to claim 5, wherein the processing circuitry is configured to perform control such that the analysis region is changeable according to a subject to be analyzed.

7. The medical image diagnosis apparatus according to claim 1, wherein the processing circuitry is configured to calculate the first similarity by setting a comparison region corresponding to a plurality of pixels in a corresponding position in each of the frames and comparing the pixels in the comparison region between frames.

8. The medical image diagnosis apparatus according to claim 7, wherein the processing circuitry is configured to perform control such that the comparison region is changeable.

9. The medical image diagnosis apparatus according to claim 1, wherein the processing circuitry is configured to
calculate the second similarity with respect to each of a plurality of positions and
generate an image representing a distribution of the second similarities and output the image.

10. The medical image diagnosis apparatus according to claim 1, wherein the processing circuitry is configured to
calculate the second similarity with respect to each of a plurality of positions and a plurality of frames and
generate an image representing a distribution of the second similarities with respect to each of the frames and output the images.

11. The medical image diagnosis apparatus according to claim 1, wherein the processing circuitry is configured to calculate the first similarity with respect to a new frame every time the signals are acquired and calculate the second similarity based on the first similarities that are calculated with respect to a given number of frames from the new frame.

12. The medical image diagnosis apparatus according to claim 1, wherein the processing circuitry is configured to calculate the first similarity with respect to a new frame every time the signals are acquired and, every time the first similarities are calculated with respect to a given number of frames, calculate the second similarity based on the first similarities that are calculated with respect to the given number of frames.

13. The medical image diagnosis apparatus according to claim 11, wherein the processing circuitry is configured to perform control such that the given number of frames is changeable according to a subject to be analyzed.

14. A medical image processing apparatus comprising a processing circuitry configured to
calculate a first similarity representing a similarity of signals that are acquired from a subject over time between frames with respect to each of a plurality of frames and a plurality of positions and calculate a second similarity representing a similarity of change of the first similarity over time between the positions, and
output the second similarity.

15. A medical image processing method comprising:
calculating a first similarity representing a similarity of signals that are acquired from a subject over time between frames with respect to each of a plurality of frames and a plurality of positions and calculate a second similarity representing a similarity of change of the first similarity over time between the positions; and
outputting the second similarity.
